(19) United States
(12) Patent Application Publication
(10) Pub. No.: US 2006/0150286 A1
(43) Pub. Date: Jul. 6, 2006

(54) Gene suppression in transgenic plants using multiple constructs

(76) Inventors: Shihshieh Huang, Stonington, CT (US); Larry Gilbertson, Chesterfield, MO (US)

Correspondence Address: MONSANTO COMPANY, 800 N. LINDBERGH BLVD., ATTENTION: GAIL P. WUELLNER, IP PARALEGAL, (E2NA), ST. LOUIS, MO, 63167, US

(21) Appl. No.: 11/311,892

(22) Filed: Dec. 19, 2005

Related U.S. Application Data
(60) Provisional application No. 60/638,491, filed on Dec. 23, 2004.

(51) Int. Cl. A01H 5/00; A01H 1/00; C07H 21/04

(52) U.S. Cl. 800/294; 800/320.1; 536/23.2

(57) ABSTRACT

Methods of gene suppression comprise transforming eukaryotic cells with multiple copies of gene suppression cassettes which are assembled into a DNA construct with promoters of each cassette at the ends of the construct.

### REFERENCE TO RELATED APPLICATIONS

This application claims priority to U.S. Provisional application No.60/638,491, filed Dec. 23, 2004, incorporated herein by reference.

### FIELD OF THE INVENTION

Disclosed herein are plasmids and methods useful in gene suppression and transgenic plants containing DNA transferred using such plasmids and methods.

### BACKGROUND

Redenbaugh et al. in “Safety Assessment of Genetically Engineered Fruits and Vegetables—A case study of the Flavr Savr™ Tomato”, CRC Press, Inc. (1992) disclosed introducing an anti-sense DNA construct into a tomato genome by Agrobacterium transformation to produce gene silencing of the polygalacturonase (PG) gene. A common characteristic of transferred DNA (T-DNA) in transgenic plants exhibiting the desired trait was two or more T-DNA regions or fragments inserted in a head to head and/or tail to tail arrangement consistent with a report by Jorgensen et al. Mol.Gen. Genet. 207:471-477 (1987) that multiple copies of the T-DNA are often transferred to and integrated into the genome of a single cell; and, when this occurs, the T-DNAs are predominately organized in inverted repeat structures in plants transformed with Agrobacterium. With reference to FIG. 1 and Table 1 tomato was transformed with a plasmid containing the anti-sense construct (FIG. 1a) comprising a full-length PG cDNA in the anti-sense orientation between an “enhanced” 35S CaMV promoter and the 3′ region of the Agrobacterium tml gene and an artificial kan marker gene. This construct was used for commercial-scale transformations of several inbred tomato lines as part of the development and marketing of Flavr Savr™ tomatoes by Calgene in 1994. Tomato lines denoted 501, 502, 7B, 22B and 28B were transformed with pCGN1436 using disarmed Agrobacterium tumefaciens. Events were selected based primarily on phenotype, i.e. low PG enzyme activity in ripe fruit. Approximately 150 transgenic event plants were produced for each inbred and 573 plants with ripe fruit were assayed for PG levels. Between 14-25% of those events across all tomato lines had PG activity lowered by 95% or greater and resulted in a total of 103 events. Of those plants, 84 had enough seed for kanamycin germination assays to determine segregation ratios and 27 events (representing between 3-10 events for each inbred) segregated 3:1 for the kan gene. Based on preliminary southern analysis, only about 40% of the 27 events with 3:1 segregation ratios clearly appeared to have the PGAS gene and kan gene inserted at a single physical locus. Eight of those events were chosen for detailed molecular analysis of T-DNA insert structures based on the availability of homozygous lines. The results of those analyses are shown in FIG. 1b-d, with the finding that all 8 events across the inbred lines had T-DNA inserts containing inverted repeat elements. The data were consistent with event 501-1001 having only a single T-DNA insert, but with the tml 3′ region present as an inverted repeat as illustrated in FIG. 1b. Six events appeared to contain two T-DNA regions in a “tail to tail” arrangement as illustrated in FIG. 1c and event 501-1035 had 3 inserts integrated in a manner illustrated in FIG. 1d.

| Element | Reference |
|---|---|
| Left border | from T-DNA of pTiA6, Barker |
| | et al., Plant Mol. Biol. |
| | 2: 335-350 (1983) |
| Mas 5′ promoter | from mannopine synthase |
| | gene, Barker et al., ibid |
| Npt II | neomycin phosphotransferase |
| | gene from transposon Tn5, |
| | Jorgenson, Mol. Gen. 177: |
| | 65 (1979) |
| Mas 3′ | polyadenylation region from |
| | mannopine synthase gene |
| | Barker et al., ibid |
| Double CaMV35S | Gardner et al., Nucl. Acids |
| promoter | Res. 9: 2871-2888 (1981) |
| Anti-sense PG | full length of polygalacturonase |
| | cDNA in anti-sense orientation, |
| | Sheehy et al. Proc. Natl. Acad. |
| | Sci. USA. 85: 8805-8809 |
| Tml 3′ | polyadenylation region of |
| | tml gene from pTiA6, Barker et |
| | al. ibid |
| Right border | with overdrive t-strand |
| | enhancer element, McBride et al. |
| | Plant, Mol. Biol. 14: |
| | 269-276 (1990) |

Northern analysis of the 8 selected events demonstrated no correlation between PG anti-sense RNA levels and the efficacy of PG gene silencing. A range of PG anti-sense RNA levels were observed, ranging from easily detected amounts in one event to undetectable levels in multiple events, all of which produced the gene silenced trait of delayed ripening. Potential read-through transcripts larger in size than expected were detected for the marker kan gene and for the PG anti-sense gene. The observation that inverted repeat elements in T-DNA inserts were likely transcribed as larger than expected RNAs, albeit at low levels, supports the thesis that PG mRNA reductions were due to RNAi induced by the production of RNA capable of forming dsRNA.

The structure of anti-sense insert illustrated in FIG. 1b with inverted repeat of 3′ tml (sense followed by anti-sense) is very similar to the sense construct utilized for gene silencing by Brummell et al. as disclosed in Plant Journal, 33, 793-800 (2003) using 3′ nos element (anti-sense followed by sense) as an inverted repeat. In each case a 3′ hairpin loop could be formed and used as primer for RNA-dependent RNA polymerase and the formation of dsRNA sequences of the target RNA.

The discovery of inverted repeats of inserted T-DNA illustrated in FIG. 1c and as an element of FIG. 1c, suggested increasing the efficacy of transformation with anti-sense DNA constructs by directly transforming with the inverted repeat in the plasmid.

Yet, the presence of inverted repeats in plasmids has been believed to be problematic when inside bacteria, e.g. E. coli, which interfere with plasmid maintenance, resulting in plasmid instability. The following described invention provides the potential advantages of employing inverted repeat elements in a transformation construct without the disadvantage of adjacent inverted repeats in bacteria.

A single expression cassette containing inverted repeats of sequences from a target gene may not be effective for gene suppression in desired plant tissue. For instance, the CaMV 35S promoter is typically denoted as “constitutive”, but is does not express well in pollen. The “constitutive” rice actin 1 promoter expresses well in pollen but not as well in leaves. The following described invention provides advantages of gene suppression in multiple plant tissues not afforded by use of a single cassette with a single promoter.

### SUMMARY OF THE INVENTION

This invention provides an improved method of gene suppression comprising transforming eukaryotic cells with multiple copies of gene suppression constructs located adjacent to each other on a plasmid. In one aspect of the invention the multiple copies of gene suppression constructs can be multiple adjacent copies of anti-sense gene suppression constructs; in another aspect they can be multiple adjacent copies of sense (co-suppression) gene suppression constructs. More particularly the method comprises inserting into a plasmid for Agrobacterium-mediated transformation a cassette for expressing sense (or anti-sense) DNA from a gene targeted for suppression adjacent to a second cassette for expressing the same sense (or anti-sense) DNA.

A characteristic of the invention is variation in regulatory elements in the cassettes, i.e. the promoter regulatory elements and/or the polyadenylation regulatory elements. In embodiments using anti-sense cassettes, the first anti-sense expression cassette comprises a first promoter operably linked to DNA of a gene targeted for suppression in an anti-sense orientation optionally followed by a first 3′ element (e.g. comprising a polyadenylation signal and polyadenylation site); and, the second anti-sense RNA expression cassette comprises a second promoter operably linked to said DNA of a gene targeted for suppression in an anti-sense orientation optionally followed by a second 3′ element. The first and second cassettes are assembled into a DNA construct in a tail-to-tail configuration so that the promoters are at the ends of the assembled construct bounding transcribable DNA of the gene targeted for suppression and, when 3′ elements are used, the 3′ elements are (a) contiguous or (b) adjacent to the promoters either between the promoters and the transcribable DNA or at the extreme regions of the assembly. At a minimum the first and second promoters are different. First and second 3′ elements can also be are different.

The method further comprises transforming eukaryotic cells by transferring a DNA construct with such assembled first and second cassettes from a plasmid by Agrobacterium-mediated transformation. A transgenic organism is regenerated from cells transformed with the first and second cassettes; and, a trait resulting from suppression of the level of protein encoded by said DSA of a gene targeted for suppression is measured in the transgenic organism.

In aspects of the method promoters can include well-know promoters that are functional in plants including Agrobacterium nopaline synthase (nos) promoter, Agrobacterium octopine synthase (ocs) promoter, the cauliflower mosaic virus promoter (CaMV 35S), figwort mosaic virus promoter (FMV), maize RS81 promoter, rice actin promoter, maize RS324 promoter, maize PR-1 promoter, maize A3 promoter, gamma coixin B32 endosperm-specific promoter, maize L3 oleosin embryo-specific promoter, rd29a promoter, and any of the other well-know promoters useful in plant gene expression.

In aspects of the method the 3′ elements are selected from the group consisting of the well-known 3′ elements, e.g. Agrobacterium gene 3′ elements such as nos 3′, tml 3′, tmr 3′, tms 3′, ocs 3′, tr7 3′ and plant gene 3′ elements such as wheat (Triticum aesevitum) heat shock protein 17 (Hsp17) 3′, a wheat ubiquitin gene 3′, a wheat fructose-1,6-biphosphatase gene 3′, a rice glutelin gene 3′, a rice lactate dehydrogenase gene 3′, a rice beta-tubulin gene 3′, a pea (Pisum sativum) ribulose biphosphate carboxylase gene (rbs) 3′, and 3′ elements from other genes within the host plant.

In other aspects of the method at least one of the multiple cassettes comprises a marker gene, e.g. an herbicide marker gene that provides resistance to glyphosate (aroA or EPSPS) or glufosinate (pat or bar); a bacteriocide marker gene that provides resistance to kanamycin (npt II), gentamycin (aac 3), hygromycin (aph IV), streptomycin and spectinomycin (aadA), or ampicilin (amp); or a screenable marker such as a luciferase (luc) or a fluorescent protein (gfp) or a beta-glucuronidase (uidA). The length of the DNA of a gene targeted for suppression can be any length, but preferably at least 21 nucleotides in length.

Another aspect of the invention provides a plasmid for Agrobacterium-mediated transformation comprising such a first cassette for expressing sense (or anti-sense) DNA from a gene targeted for suppression adjacent to such a second cassette for expressing the same DNA, where the cassettes are assembled so that the different 3′ untranslated regions are contiguous. In many cases the cassettes and at least one marker cassette are located between left and right T-DNA borders on the plasmid.

In a preferred aspect of the invention a transgenic corn plant contains a DNA construct with adjacent cassettes for anti-sense suppression of the lysine ketoglutarate reductase gene using an endosperm specific promoter in one cassette and an embryo specific promoter in the other cassette.

### BRIEF DESCRIPTION OF THE DRAWINGS

FIGS. 1 and 2 illustrate DNA constructs.

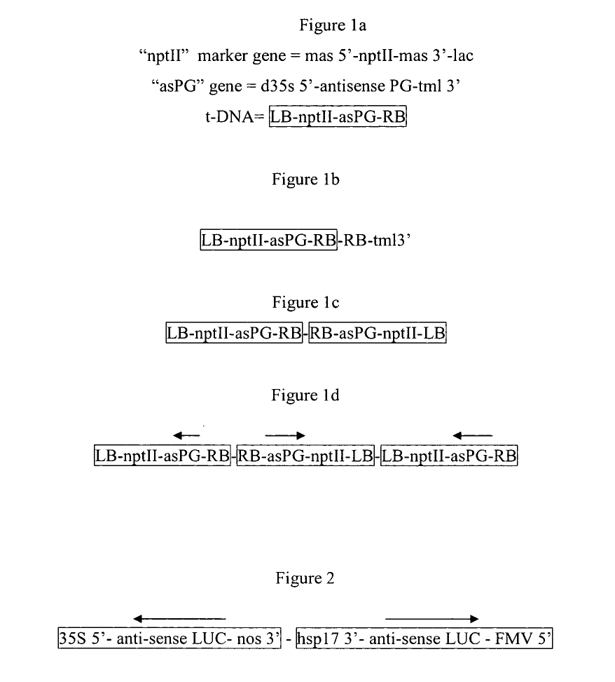

### DETAILED DESCRIPTION OF THE INVENTION

As used herein “cassette” means a combination of DNA elements normally associated with the expression of protein from a gene and comprises at least (a) DNA for initiating transcription such as a promoter element, (b) DNA coding for a protein such as cDNA or genomic DNA comprising exons and introns, and (c) DNA for splicing 3′ RNA from transcribed RNA after coding sequence and adding a polyA tail such as a 3′ element containing a polyadenylation site. Typically, when the DNA coding for a protein is in a sense orientation, the transcribed RNA can be translated to express protein or, in some cases, for sense co-suppression. When the DNA coding for protein is in an anti-sense orientation, the transcribed RNA can be involved in a gene suppression mechanism. For instance, to promote gene suppression anti-sense DNA typically corresponds to DNA that is transcribed to mRNA upstream of a polyadenylation site. Thus, an “anti-sense cassette” means a combination of DNA elements comprising a promoter operably linked to anti-sense oriented DNA from a gene targeted for suppression and a 3′ element. Although common, it is not critical that the 3′ element contain a polyadenylation site. What is important in either adjacent sense cassettes or adjacent anti-sense cassettes is that adjacent 3′ elements are distinct, i.e. transcribed RNA from adjacent 3′ elements is are not capable of hybridizing to from double-stranded RNA or being readily excised from a plasmid in E.coli.

Recombinant DNA constructs, e.g. the cassettes of this invention, can be readily prepared by those skilled in the art using commercially available materials and well-known, published methods. When multiple genes are targeted for suppression, polycistronic DNA elements can be fabricated as illustrated and disclosed in U.S. application Ser. No. 10/465,800, incorporated herein by reference. A useful technology for building DNA constructs and vectors for transformation is the GATEWAY™ cloning technology (available from Invitrogen Life Technologies, Carlsbad, Calif.) uses the site specific recombinase LR cloning reaction of the Integrase att system from bacterophage lambda vector construction, instead of restriction endonucleases and ligases. The LR cloning reaction is disclosed in U.S. Pat. Nos. 5,888,732 and 6,277,608, U.S. Patent Application Publications 2001283529, 2001282319, 20020007051, and 20040115642, all of which are incorporated herein by reference. The GATEWAY™ Cloning Technology Instruction Manual which is also supplied by Invitrogen also provides concise directions for routine cloning of any desired DNA into a vector comprising operable plant expression elements.

An alternative vector fabrication method employs ligation-independent cloning as disclosed by Aslandis, C. et al., Nucleic Acids Res., 18, 6069-6074, 1990 and Rashtchian, A. et al., Biochem., 206, 91-97,1992 where a DNA fragment with single-stranded 5′ and 3′ ends are ligated into a desired vector which can then be amplified in vivo.

Numerous promoters that are active in plant cells have been described in the literature. These include promoters present in plant genomes as well as promoters from other sources, including nopaline synthase (NOS) promoter and octopine synthase (OCS) promoters carried on tumor-inducing plasmids of Agrobacterium tumefaciens, caulimovirus promoters such as the cauliflower mosaic virus or figwort mosaic virus promoters. For instance, see U.S. Pat. Nos. 5,858,742 and 5,322,938 which disclose versions of the constitutive promoter derived from cauliflower mosaic virus (CaMV35S), U.S. Pat. No. 5,378,619 which discloses a Figwort Mosaic Virus (FMV) 35S promoter, U.S. Pat. No. 6,437,217 which discloses a maize RS81 promoter, U.S. Pat. No. 5,641,876 which discloses a rice actin promoter, U.S. Pat. No. 6,426,446 which discloses a maize RS324 promoter, U.S. Pat. No. 6,429,362 which discloses a maize PR-1 promoter, U.S. Pat. No. 6,232,526 which discloses a maize A3 promoter, U.S. Pat. No. 6,177,611 which discloses constitutive maize promoters, U.S. Pat. No. 6,433,252 which discloses a maize L3 oleosin promoter, U.S. Pat. No. 6,429,357 which discloses a rice actin 2 promoter and intron, U.S. Pat. No. 5,837,848 which discloses a root specific promoter, U.S. Pat. No. 6,084,089 which discloses cold inducible promoters, U.S. Pat. No. 6,294,714 which discloses light inducible promoters, U.S. Pat. No. 6,140,078 which discloses salt inducible promoters, U.S. Pat. No. 6,252,138 which discloses pathogen inducible promoters, U.S. Pat. No. 6,175,060 which discloses phosphorus deficiency inducible promoters, U.S. Patent Application Publication 2002/0192813A1 which discloses 5′, 3′ and intron elements useful in the design of effective plant expression vectors, U.S. patent application Ser. No. 09/078,972 which discloses a coixin promoter, U.S. patent application Ser. No. 09/757,089 which discloses a maize chloroplast aldolase promoter, and U.S. patent application Ser. No. 10/739,565 which discloses water-deficit inducible promoters, all of which are incorporated herein by reference. These and numerous other promoters that function in plant cells are known to those skilled in the art and available for use in recombinant polynucleotides of the present invention to provide for expression of desired genes in transgenic plant cells.

In aspects of the method the 3′ elements are selected from the group consisting of the well-known 3′ elements from Agrobacterium tumefaciens genes such as nos 3′, tml 3′, tmr 3′, tms 3′, ocs 3′, tr7 3′, e.g. disclosed in U.S. Pat. No. 6,090,627, incorporated herein by reference; 3′ elements from plant genes such as wheat (Triticum aesevitum) heat shock protein 17 (Hsp17 3′), a wheat ubiquitin gene, a wheat fructose-1,6-biphosphatase gene, a rice glutelin gene a rice lactate dehydrogenase gene and a rice beta-tubulin gene, all of which are disclosed in U.S. published patent application 2002/0192813 A1, incorporated herein by reference; and the pea (Pisum sativum) ribulose biphosphate carboxylase gene (rbs 3′), and 3′ elements from the genes within the host plant.

Furthermore, the promoters may be altered to contain multiple “enhancer sequences” to assist in elevating gene expression. Such enhancers are known in the art. By including an enhancer sequence with such constructs, the expression of the selected protein may be enhanced. These enhancers often are found 5′ to the start of transcription in a promoter that functions in eukaryotic cells, but can often be inserted in the forward or reverse orientation 5′ or 3′ to the coding sequence. In some instances, these 5′ enhancing elements are introns. Particularly useful enhancers are the 5′ introns of the rice actin 1 and rice actin 2 genes, the maize alcohol dehydrogenase gene and the maize shrunken 1 gene.

In some aspects of the invention it is preferred that the promoter element in the DNA construct be capable of causing sufficient expression to result in the production of an effective amount of a polypeptide in water deficit conditions. Such promoters can be identified and isolated from the regulatory region of plant genes that are over expressed in water deficit conditions. Specific water-deficit-inducible promoters for use in this invention are derived from the 5′ regulatory region of genes identified as a heat shock protein 17.5 gene (HSP17.5), an HVA22 gene (HVA22), a Rab17 gene and a cinnamic acid 4-hydroxylase (CA4H) gene (CA4H) of Zea mays. Such water-deficit-inducible promoters are disclosed in U.S. application Ser. No.10/739,565, incorporated herein by reference.

In other aspects of the invention, sufficient expression in plant seed tissues is desired to effect improvements in seed composition. Exemplary promoters for use for seed composition modification include promoters from seed genes such as napin (U.S. Pat. No. 5,420,034), maize L3 oleosin (U.S. Pat. No. 6,433,252), zein Z27 (Russell et al. (1997) Transgenic Res. 6(2):157-166), globulin 1 (Belanger et al (1991) Genetics 129:863-872), glutelin 1 (Russell (1997) supra), and peroxiredoxin antioxidant (Per1) (Stacy et al. (1996) Plant Mol Biol. 31(6):1205-1216).

In still other aspects of the invention, preferential expression in plant green tissues is desired. Promoters of interest for such uses include those from genes such as SSU (Fischhoff et al. (1992) Plant Mol Biol. 20:81-93), aldolase and pyruvate orthophosphate dikinase (PPDK) (Taniguchi et al. (2000) Plant Cell Physiol. 41(1):42-48).

In practice DNA is introduced into only a small percentage of target cells in any one transformation experiment. Marker genes are used to provide an efficient system for identification of those cells that are stably transformed by receiving and integrating a transgenic DNA construct into their genomes. Preferred marker genes provide selective markers which confer resistance to a selective agent, such as an antibiotic or herbicide. Any of the herbicides to which plants of this invention may be resistant are useful agents for selective markers. Potentially transformed cells are exposed to the selective agent. In the population of surviving cells will be those cells where, generally, the resistance-conferring gene is integrated and expressed at sufficient levels to permit cell survival. Cells may be tested further to confirm stable integration of the exogenous DNA. Commonly used selective marker genes include those conferring resistance to antibiotics such as kanamycin (nptII), hygromycin B (aph IV) and gentamycin (aac3 and aacC4) or resistance to herbicides such as glufosinate (bar or pat) and glyphosate (EPSPS). Examples of such selectable are illustrated in U.S. Pat. Nos. 5,550,318; 5,633,435; 5,780,708 and 6,118,047, all of which are incorporated herein by reference. Screenable markers which provide an ability to visually identify transformants can also be employed, e.g., a gene expressing a colored or fluorescent protein such as a luciferase or green fluorescent protein (GFP) or a gene expressing a beta-glucuronidase or uidA gene (GUS) for which various chromogenic substrates are known.

Plant Transformation Methods

Numerous methods for transforming plant cells with recombinant DNA are known in the art and may be used in the present invention. Two commonly used methods for plant transformation are Agrobacterium-mediated transformation and microprojectile bombardment. Microprojectile bombardment methods are illustrated in U.S. Pat. No. 5,015,580 (soybean); U.S. Pat. No. 5,550,318 (corn); U.S. Pat. No. 5,538,880 (corn); U.S. Pat. No. 5,914,451 (soybean); U.S. Pat. No. 6,160,208 (corn); U.S. Pat. No. 6,399,861 (corn) and U.S. Pat. No. 6,153,812 (wheat) and Agrobacterium-mediated transformation is described in U.S. Pat. No. 5,159,135 (cotton); U.S. Pat. No. 5,824,877 (soybean); U.S. Pat. No. 5,591,616 (corn); and U.S. Pat. No. 6,384,301 (soybean), all of which are incorporated herein by reference. For Agrobacterium tumefaciens based plant transformation system, additional elements present on transformation constructs will include T-DNA left and right border sequences to facilitate incorporation of the recombinant polynucleotide into the plant genome.

In general it is useful to introduce recombinant DNA randomly, i.e. at a non-specific location, in the genome of a target plant line. In special cases it may be useful to target recombinant DNA insertion in order to achieve site-specific integration, e.g. to replace an existing gene in the genome, to use an existing promoter in the plant genome, or to insert a recombinant polynucleotide at a predetermined site known to be active for gene expression. Several site specific recombination systems exist which are known to function implants include cre-lox as disclosed in U.S. Pat. No. 4,959,317 and FLP-FRT as disclosed in U.S. Pat. No. 5,527,695, both incorporated herein by reference.

Transformation methods of this invention are preferably practiced in tissue culture on media and in a controlled environment. “Media” refers to the numerous nutrient mixtures that are used to grow cells in vitro, that is, outside of the intact living organism. Recipient cell targets include, but are not limited to, meristem cells, callus, immature embryos and gametic cells such as microspores, pollen, sperm and egg cells. It is contemplated that any cell from which a fertile plant may be regenerated is useful as a recipient cell. Callus may be initiated from tissue sources including, but not limited to, immature embryos, seedling apical meristems, microspores and the like. Cells capable of proliferating as callus are also recipient cells for genetic transformation. Practical transformation methods and materials for making transgenic plants of this invention, e.g. various media and recipient target cells, transformation of immature embryos and subsequent regeneration of fertile transgenic plants are disclosed in U.S. Pat. Nos. 6,194,636 and 6,232,526 and U.S. patent application Ser. No. 09/757,089, which are incorporated herein by reference.

The seeds of transgenic plants can be harvested from fertile transgenic plants and be used to grow progeny generations of transformed plants of this invention including hybrid plants line comprising the recombinant DNA construct expressing an agent for genes suppression.

In addition to direct transformation of a plant with a recombinant DNA construct, transgenic plants can be prepared by crossing a first plant having a recombinant DNA construct with a second plant lacking the construct. For example, recombinant DNA for gene suppression can be introduced into first plant line that is amenable to transformation to produce a transgenic plant which can be crossed with a second plant line to introgress the recombinant DNA for gene suppression into the second plant line.

A transgenic plant with recombinant DNA effecting gene suppression can be crossed with transgenic plant line having other recombinant DNA that confers another trait, e.g. yield improvement, herbicide resistance or pest resistance to produce progeny plants having recombinant DNA that confers both gene suppression and the other trait. Typically, in such breeding for combining traits the transgenic plant donating the additional trait is a male line and the transgenic plant carrying the base traits is the female line. The progeny of this cross will segregate such that some of the plants will carry the DNA for both parental traits and some will carry DNA for one parental trait; such plants can be identified by markers associated with parental recombinant DNA Progeny plants carrying DNA for both parental traits can be crossed back into the female parent line multiple times, e.g. usually 6 to 8 generations, to produce a progeny plant with substantially the same genotype as one original transgenic parental line but for the recombinant DNA of the other transgenic parental line.

### EXAMPLE 1

This example illustrates a method of this invention. With reference to FIG. 2 two cassettes are prepared for anti-sense suppression of luciferase in an organism expressing luciferase. A first luciferase anti-sense cassette comprises CaMV 35S promoter (35S 3′) operably linked to an anti-sense segment of firefly luciferase coding DNA (anti-sense LUC) and nos 3′ element. A second luciferase anti-sense cassette comprises a FMV promoter (FMV 5′) operably linked to the same anti-sense segment of firefly luciferase coding DNA and a wheat heat shock protein 3′ element (hsp 3′). The anti-sense cassettes are assembled in an transformation plasmid inverted with respect to each other with the respective 3′ elements being contiguous. Surprisingly, the assembled cassettes are not prone to excision when the plasmid is inserted into common strains of E. coli. The plasmid is co-transformed into a plant cell along with a two plasmids capable of expressing the firefly luciferase and Renilla luciferase genes, the latter serving as a baseline control against which firefly luciferase expression is normalized. Thus, the ratio of firefly luciferase to Renilla luciferase expression is a measurement of the level of suppression of the firefly luciferase gene. As compared to plant cells transformed with a single copy of either of the firefly luciferase anti-sense cassettes, the multiple cassettes exhibit a higher level of firefly luciferase suppression in transgenic plant cells.

### EXAMPLE 2

This example illustrates a construct useful for selective gene suppression in plant tissues. A first anti-sense gene suppression construct was prepared comprising a corn plant endosperm specific promoter B32 (nucleotides 848 through 1259 of GenBank accession number X70153, see also Hartings et al (1990) Plant Mol. Biol., 14:1031-1040) operably linked to transcribable DNA consisting of about 500 base pairs of the LKR domain of a maize lysine ketoglutarate reductase/saccharopine dehydrogenase gene (LKR/SDH) in first segment in an anti-sense orientation linked to a second segment in a sense orientation. Because LKR is a lysine catabolite, its suppression resulted in increased lysine. A second anti-sense gene suppression construct was prepared essentially the same as the first anti-sense gene suppression construct except that the promoter was replaced with a corn plant embryo specific promoter L3 oleosin (see U. S. Pat. No. 6,433,252). A third gene suppression construct according to this invention was prepared by linking a B32 promoter that used in the first construct to the 3′ end of the second construct providing a construct with opposing promoters operably linked to an anti-sense oriented segment of DNA from the gene targeted for suppression. In one alternative embodiment the gene suppression construct of this invention is prepared from the second anti-sense gene suppression construct by replacing the 3′ regulatory region that provides a polyadenylaiton signal and site with the B32 promoter inserted in an opposite orientation to the L3 promoter at the opposing end of the construct. In another alternative embedment the construct of this invention is prepared by adding the B32 promoter downstream of the 3′ regulatory region and in an opposite orientation to the L3 promoter at the opposing end of the construct; optionally a second 3′ regulatory region is inserted between the L3 promoter and the transcribable DNA. In yet another embodiment the construct of this invention is prepared by locating 3′ regulatory regions at the external regions of the construct where each 3′ regulatory region is oriented to the promoters at the opposing end of the construct. In still another embodiment two anti-sense constructs are assembled in a tail-to-tail orientation providing a construct counded by the respective promoters.

Plasmids suitable for Agrobacterium-mediated plant transformation were prepared using each of (a) the first anti-sense gene suppression construct with the B32 promoter, (b) the second ant-sense gene suppression construct with the L3 promote and (c) a gene suppression construct of this invention with a B32 and an L3 promoter at opposing ends of the construct and in opposite orientations. Each construct was inserted into a plasmid for binary vector of an Agrobacterium-mediated transformation system between left and right T-DNA borders and next to a selectable marker cassette for expressing an aroA gene from A. tumefaciens. Each plasmid was inserted into maize callus by Agrobacterium-mediated transformation. Events were selected as being resistance to glyphosate herbicide and grown into transgenic maize plants to produce F1 seed. Mature seeds from each event is analyzed to determine success of transformation and suppression of LKR. The mature transgenic seeds are dissected to extract protein for Western analysis. Seed from transgenic maize plants shows reduction in LKR and increased lysine as compared to wild type. The first construct with the endosperm specific promoter provides seed with about 1000 ppm of free lysine; LKR reduction is essentially observed only in endosperm tissue. The second construct with the embryo specific promoter provides seed with about 300 ppm of free lysine; LKR reduction is essentially observed only in embryo tissue. Because lysine is believed to travel between embyo and endosperm, concurrent suppression of LKR in both embryo and endosperm tissues using the construct of this invention provides seed with higher values of free lysine than the additive effect from suppression in one tissue alone, e.g. greater than 1300 ppm.

What is claimed is:

1. A method of gene suppression comprising transforming eukaryotic cells with multiple copies of gene suppression cassettes, wherein said method comprises
(a) assembling a DNA construct comprising a first gene suppression cassette adjacent to a second gene suppression cassette, wherein said first cassette comprises a first promoter operably linked to DNA of a gene targeted for suppression in either a sense or an anti-sense orientation, wherein said second cassette comprises a second promoter operably linked to at least a part of said DNA in the same orientation, wherein said first and second cassettes are assembled so that the promoter elements are at the ends of the construct,
(b) transforming said eukaryotic cells by transferring an assembly of said first and second cassettes into said cells,
(c) regenerating a transgenic organism from cells transformed with said assembly, and
(d) whereby a trait resulting from suppression of the level of protein encoded by said DNA of a gene targeted for suppression can be observed in said organism.

2. A method of claim 1 wherein said organism is a plant.

3. A method of claim 1 wherein said DNAs in said assembly of cassettes are in an anti-sense orientation or said DNAs in said assembly of cassettes are a sense orientation.

4. A method of claim 1 wherein said first and second promoters are different.

5. A method of claim 4 wherein said first promoter is a plant seed embryo specific promoter and said second promoter is a plant seed endosperm specific promoter.

6. A method of claim 4 wherein said promoters are selected from the group consisting of a nos promoter, an acs promoter a CaMV 35S promoter, a rice actin promoter, a B32 promoter and an L3 promoter.

7. A method of claim 1 wherein said cassettes further comprise distinct 3′ elements.

8. A method of claim 7 wherein said 3′ elements are selected from the group consisting of nos 3′, tml 3′, ocs 3′, tr7 3′, wheat Hsp17 3′ untranslated regions.

9. A method of claim 1 wherein at least one of said cassettes comprises a marker gene.

10. A method of claim 9 wherein said marker gene is an herbicide marker gene that provides resistance to glyphosate or glufosinate or a bacteriocide marker gene that provides resistance to kanamycin, hygromycin,streptomycin or streptinomycin

11. A method of claim 1 wherein said DNA of a gene targeted for suppression is at least in the range of 19 to 23 nucleotides in length.

12. A DNA construct comprising a first cassette adjacent to a second cassette, wherein said first cassette comprises a first promoter operably linked to DNA of a gene targeted for suppression, wherein said second cassette comprises a second promoter operably linked to DNA of a gene targeted for suppression, wherein said first and second cassettes are assembled in said construct so that the promoters are at the opposite ends of the construct.

13. A plasmid for Agrobacterium-mediated transformation of plants comprising a DNA construct of claim 12 and at least one marker cassette are located between left and right T-DNA borders.

14. A plasmid of claim 12 wherein said DNA of a gene targeted for suppression is in an anti-sense orientation in each of said first and second cassettes.

15. A transgenic plant having in its genome a DNA construct of claim 12.

16. A transgenic plant of claim 15 for gene suppression wherein said first promoter is a plant seed embryo specific promoter and a second promoter is an endosperm specific promoter.

17. A transgenic corn plant of claim 16 wherein said DNA construct suppresses the production of a lysine catabolite.
